(19) United States
(12) Patent Application Publication
(10) Pub. No.: US 2015/0353660 A1
(43) Pub. Date: Dec. 10, 2015

(54) PROCESS FOR PREPARING CATALYSTS

(71) Applicant: LANXESS Deutschland GmbH, Cologne (DE)

(72) Inventors: Pierre VANHOORNE, Monheim (DE); Hubertus MITTAG, Bitterfeld-Wolfen (DE); Reinhold KLIPPER, Cologne (DE); Rudolf WAGNER, Cologne (DE)

(21) Appl. No.: 14/725,072

(22) Filed: May 29, 2015

(30) Foreign Application Priority Data
Jun. 5, 2014 (EP) 14171224.0

(51) Int. Cl. C08F 212/08; C07C 37/20

(52) U.S. Cl. CPC C08F 212/08; C07C 37/20

(57) ABSTRACT

Catalysts having a higher total capacity and containing fewer organic impurities are provided for condensation, addition and esterification reactions, as we as a process for preparing these catalysts and for use of the catalysts for preparation of bisphenols.

### BACKGROUND

Catalysts are often used for condensation, addition and esterification reactions. One such type of reaction may include the preparation of bisphenols.

The condensation of phenols and ketones to give bisphenols plays a major role in industrial preparation processes. Bisphenol A in particular serves, inter alia, for preparation of polycarbonate and may be prepared by condensation of phenol and acetone in the presence of hydrogen chloride or polystyrenesulphonic acids as catalysts. The polystyrenesulphonic acids used may be strongly acidic cation exchangers which have to be neutralized. Frequently, this may be accomplished by adding what is called a promoter, for example a mercaptan, in reactors with vigorous stirring. However, industrial reactors having correspondingly large and extensive stirring apparatuses are rare and the mixing or homogeneous coating of the catalysts with the promoters may still be unsatisfactory,

One way of overcoming the abovementioned disadvantage is to undertake the doping in the course of the process for preparing the strongly acidic cation exchangers.

EP 0466277 A discloses, for example, a process for preparing a doped bisphenol A catalyst, in which a styrene-divinylbenzene-based strongly acidic cation exchanger is sulphonated in the presence of sulphuric acid and a halogenated swelling agent and then doped with a mercaptan promoter.

Further processes for preparing bisphenol A catalysts based, inter alia, on sulphonated styrene-divinylbenzene copolymers by means of doping with promoters are disclosed in WO2008/157025 A or DE 2164339 B.

The catalysts used essentially have inadequate purity and/or the catalyst activity is insufficient. There remains therefore a need for improved catalysts and processes for the preparation thereof to overcome the above-discussed disadvantages.

### SUMMARY

It has now been found that, surprisingly, it may be possible with the aid of the process according to the invention to prepare catalysts having a higher total capacity and containing fewer organic impurities than catalysts which are prepared by conventional preparation processes in the presence of a swelling agent.

The invention therefore provides a process for preparing a catalyst, in which
a) monomer droplets of a mixture comprising at least one monoethylenically unsaturated aromatic compound, at least one multiethylenically unsaturated compound and at least one initiator may be converted to a crosslinked bead polymer,
   and
b) the crosslinked bead polymer from step a) may be sulphonated in the presence of sulphuric acid at a temperature of 50° C. to 160° C. and the concentration of the sulphuric acid during the reaction may be at least 75% by weight and the amount of sulphuric acid used may be 70% by weight to 95% by weight and the amount of the bead polymer used may be 5% by weight to 30% by weight, based on the total amount of sulphuric acid and bead polymer used, and the sum total of the percentages by weight of sulphuric acid and bead polymer based on the amount of the reaction mixture may be >96% by weight,
   and
c) the sulphonated crosslinked bead polymers from step b) may be reacted with at least one sulphur compound from the group of thioalcohols, thioethers and thioesters or mixtures of these compounds.

### DETAILED DESCRIPTION

Crosslinked bead polymers suitable in accordance with the invention may be copolymers of at least one monoethylenically unsaturated aromatic compound and at least one multiethylenically unsaturated compound.

The monoethylenically unsaturated aromatic (=vinylaromatic) compounds used in step a) may preferably include stynene, α-methylstyrene, vinyltoluene, ethylstyrene, t-butylstyrene, chlorostyrene, bromostyrene, chloromethylstyrene or vinylnaphthalene. Also of good suitability are mixtures of these monomers. Particular preference may be given to styrene and vinyltoluene.

The multiethylenically unsaturated compounds in step a) serve as crosslinkers. The multiethylenically unsaturated compounds used in step a) may preferably be divinylbenzene, divinyltoluene, trivinylbenzene, octadiene or triallyl cyanurate. More preferably, the multiethylenically unsaturated compounds may be vinylaromatic compounds, such as especially divinylbenzene and trivinylbenzene. Very particular preference may be given to divinylbenzene. For preparation of the bead polymers, it may be possible to use technical grade qualities of divinylbenzene containing typical products such as ethylvinylbenzene as well as the isomers of divinylbenzene. According to the invention, technical grade qualities having divinylbenzene contents of 55% to 85% by weight may be of particularly good suitability. The multiethylenically unsaturated compounds can be used alone or as a mixture of various multiethylenically unsaturated compounds.

The total amount of multiethylenically unsaturated compounds for use in step a) may generally be 0.5% to 6% by weight, based on the sum total of the ethylenically unsaturated compounds. However, it may likewise be possible to use smaller or greater amounts. The total amount of multiethylenically unsaturated compounds for use in step a) may preferably be 1.5% to 5% by weight, more preferably 1%, to 4% by weight, based on the sum total of the ethylenically unsaturated compounds.

Preference may be given to using a mixture of styrene and divinylbenzene in step a).

For preparation of the crosslinked bead polymers in step a), the abovementioned ethylenically unsaturated compounds (monomers), in a further preferred embodiment of the present invention, may be polymerized in the presence of a dispersing aid using an initiator in aqueous suspension.

Dispersing aids used may preferably include natural and synthetic water-soluble polymers. Particular preference may be given to using gelatin, cellulose derivatives, starch, polyvinyl alcohol, polyvinylpyrrolidone, polyacrylic acid, polymethacrylic acid or copolymers of (meth)acrylic acid and (meth)acrylic esters. Very particular preference may be given to using gelatin and cellulose derivatives, especially cellulose esters and cellulose ethers, such as carboxymethyl cellulose, methyl cellulose, hydroxyethyl cellulose or methyl hydroxyethyl cellulose. The amount of the dispersing aids used may generally be 0.05% to 1%, preferably 0.1% to 0.5%, based on the water phase.

In step a) in the present invention, the initiators may be used in the monomer mixture. The monomer mixture refers in the present invention to the mixture of monoethylenically unsaturated aromatic compound(s) and multiethylenically unsaturated compound(s). Suitable initiators may include compounds which form free radicals with increasing temperature and dissolve in the monomer mixture. Preference may be given to using peroxy compounds, more preferably dibenzoyl peroxide, dilauryl peroxide, bis(p-chlorobenzoyl) peroxide, dicyclohexyl peroxydicarbonate or tert-amyl peroxy-2-ethylhexane, and azo compounds, more preferably 2,2′-azobis(isobutyronitrile) or 2,2′-azobis(2-methylisobutyronitrile), or else aliphatic peroxy esters, preferably tert-butyl peroxyacetate, tea-butyl peroxyisobutyrate, tert-butyl peroxypivalate, tert-butyl peroxyoctoate, tert-butyl peroxy-2-ethylhexanoate, tert-butyl peroxyneodecanoate, tert-amyl peroxypivalate, tert-amyl peroxyoctoate, tert-amyl peroxy-2-ethylhexanoate, tert-amyl peroxyneodecanoate, 2,5-bis(2-ethylhexanoylperoxy)-2,5-dimethylhexane, 2,5-dipivaloyl-2,5-dimethylhexane, 2,5-bis(2-neodecancylperoxy)-2,5-dimethylhexane, di-tert-butyl peroxyazelate or di-tert-amyl peroxyazelate.

The initiators which may be soluble in the monomer mixture may generally be used in amounts of 0.05% to 6.0% by weight, based on the sum total of the ethylenically unsaturated compounds. However, it may likewise be possible to use smaller or greater amounts. The initiators which may be soluble in the monomer mixture may preferably be used in amounts of 0.1% to 5.0% by weight, more preferably 0.2% to 2% by weight, based on the sum total of the ethylenically unsaturated compounds.

The water phase may contain a buffer system which sets the pH of the water phase to a value between 12 and 3, preferably between 10 and 4. Buffer systems of particularly good suitability contain phosphate, acetate, citrate or borate salts.

It may be advantageous to use an inhibitor dissolved in the aqueous phase. Useful inhibitors include both inorganic and organic substances. Examples of inorganic inhibitors may include nitrogen compounds such as hydroxylamine, hydrazine, sodium nitrite or potassium nitrite.

Examples of organic inhibitors may include phenolic compounds such as hydroquinone, hydroquinone monomethyl ether, resorcinol, catechol, tert-butylcatechol, condensation products of phenols with aldehydes. Further organic inhibitors may include nitrogen compounds, for example diethylhydroxylamine and isopropylhydroxylamine. Resorcinol may be a preferred inhibitor. The concentration of the inhibitor may be 5-1000 ppm, preferably 10-500 ppm, more preferably 20-250 ppm, based on the aqueous phase.

The organic phase can be dispersed into the aqueous phase as droplets by stirring or by jetting, Organic phase may be understood to mean the monomer mixture with the initiator(s),

In the conventional dispersion polymerization, the organic droplets may be produced by stirring. On the 4 litre scale, stirrer speeds of 250 to 400 rpm may typically be used.

If the droplets are produced by jetting, it may be advisable to maintain the homogeneous droplet diameter by encapsulating the organic droplets. Processes for microencapsulation of jetted organic droplets are described, for example, in EP-A 0 046 535, the content of which in relation to microencapsulation may be encompassed by the present application.

The median particle size of the optionally encapsulated monomer droplets may be 10-1000 pm, preferably 100-1000 μm.

The ratio of the organic phase to the aqueous phase may generally be 1:20 to 1:0.6, preferably 1:10 to 1:1, more preferably 1:5 to 1:1.2.

Alternatively, the organic phase, in accordance with EP-A 0 617 714, the teaching of which may be encompassed by the present application, can be added in what may be called the seed-feed method to a suspension of seed polymers which take up the organic phase, The median particle size of the seed polymers swollen with the organic phase may be 5-1200 μm, preferably 20-1000 μm. The ratio of the sum total of organic phase and seed polymer to the aqueous phase may generally be 1:20 to 1:0.6, preferably 1:10 to 1:1, more preferably 1:5 to 1:1.2.

The polymerization of the monomers may be conducted at elevated temperature. The polymerization temperature may be guided by the breakdown temperature of the initiator and may typically be 50 to 150° C., preferably 60 to 130° C. The polymerization time may be 30 minutes to 24 hours, preferably 2 to 15 hours.

At the end of the polymerization, the crosslinked bead polymers may be separated from the aqueous phase, preferably on a suction filter, and optionally dried.

Step a) of the process according to the invention may preferably be conducted in the absence of compounds selected from toluene, ethylbenzene, xylene, cyclohexane, octane, isooctane, decane, dodecane, isododecane, methyl isobutyl ketone, ethyl acetate, butyl acetate, dibutyl phthalate, n-butanol, 4-methyl-2-pentanol, n-octanol, and perogens. The use of “preferably in the absence of” in the context of the invention means that the amount in the reaction mixture may at most be 1% by weight to 4% by weight, very especially preferably <1% by weight, and even further preferably that none is present.

The crosslinked bead polymers prepared in step a) may be sulphonated in step b). According to the invention, the sulphonation in step b) may be conducted at a concentration of the sulphuric acid of at least 75% by weight. Preferably, the sulphonation may be effected in such a way that, during the reaction, the concentration of the sulphuric acid may be between 80% by weight and 98% by weight. Typically, in order to achieve these concentrations during the sulphonation, sulphuric acids having a concentration between 80% by weight and 100% by weight may be used. If the sulphuric acid were to be used, for example, in a concentration of 80% by weight, the remainder would be water in a concentration of 20% by weight. Alternatively, it may be possible to use sulphuric acids having lower concentrations and in that case to increase the concentration further by addition of sulphur trioxide. Accordingly, it would also be possible to use sulphuric acid of a concentration of 60% by weight and then to add sulphur trioxide, such that the concentration of the sulphuric acid during the sulphonation reaction may be at least 75% by weight, preferably 80% by weight to 100% by weight. Preference may be given to adding no additional sulphur trioxide to the sulphuric acid in step b).

Preferably, the sulphuric acid used in step b) may have a concentration of 92% by weight to 99% by weight. More preferably, the concentration during the sulphonation reaction in step b) may be between 89% by weight and 96% by weight when using a sulphuric acid having a starting concentration of 92% by weight to 99% by weight.

It may be advantageous in step b) to set the necessary acid concentration by mixing sulphuric acid of a higher concentration and a lower concentration, in which case the sulphuric acid having the lower concentration used may be recovered sulphuric acid from earlier sulphonation reactions. The mixing of the sulphuric acid can be effected in the sulphonation reactor in the presence of the bead polymer to be sulphonated, such that the heat of mixing which occurs leads to an increase in the temperature of the reaction mixture.

In step b), the sulphuric add should be used in an amount of 70% by weight to 95% by weight and the bead polymer in an amount of 5% by weight to 30% by weight, where the sum total of the percentages by weight of the sulphuric acid and the bead polymer based on the amount of the reaction mixture may be >96% by weight. The remainder to 100% by weight could, for example, be further organic solvents or unpolymerized monomer residues. Preferably, the sulphonating agent may be used in step b) in an amount of 70% by weight to 95% by weight in a concentration of 92% by weight to 99% by weight, in which case the amount of the bead polymer may be between 5% by weight and 30% by weight and the sum total of the percentages by weight of the sulphuric add and the bead polymer based on the amount of the reaction mixture may be >96% by weight. Preferably, the sum total of the percentages by weight of the sulphuric acid and the bead polymer based on the amount of the reaction mixture may be >98% by weight, most preferably 100% by weight.

Step b) of the process according to the invention may preferably be conducted in the absence of a swelling agent, such as especially 1,2-dichloroethane. Swelling agents may include all organic aliphatic or aromatic solvents. More preferably, swelling agents in the context of the invention may include 1,2-dichloroethane, methylene chloride and dichlorobenzene. “Preferably in the absence of a swelling agent” in the context of the invention means that the amount of swelling agents in the reaction mixture may be at most between 1% by weight and <4% by weight, very especially preferably <1% by weight, and even further preferably that no swelling agent may be present. It has been found that the bead polymers during the sulphonation in step b) have a diameter between 5% and 15% less than during the sulphonation in the presence of a swelling agent

The temperature in the sulphonation in step b) may preferably be 90° C. to 140° C.

It may be advantageous to employ a temperature programme in step b), in which the sulphonation may be commenced in a first reaction step at a first temperature and continued in a second reaction step at a higher temperature.

Preferably, the reaction mixture may first be stirred at 90° C. to 110° C. for between 10 min and 60 min and then heated to a temperature of 120° C. to 140° C., and heated at constant temperature for a further 3 to 7 hours.

In the sulphonation in step b), the reaction mixture may be stirred. This can be done by means of various stirrer types, such as paddle stirrers, anchor stirrers, gate stirrers or turbine stirrers.

The duration of the sulphonation reaction in step b) may generally be several hours, preferably between 1 and 24 h, more preferably between 2 and 16 h, most preferably between 3 and 12 h.

After the sulphonation in step b), the reaction mixture of sulphonation product and residual acid can first be cooled to room temperature and then diluted with sulphuric acid of decreasing concentrations and then with water.

The sulphonated crosslinked bead polymers from step b) of the process according to the invention may include strongly acidic cation exchangers which may optionally be purified further before they are used in step c). The purification can be conducted with deionized water at temperatures of 70-180° C., preferably 70-130° C., more preferably 70° C. to 100° C. Preferably, the sulphonated crosslinked bead polymers from step b) may first be purified before they are converted further in step c).

Thioalcohols used in step c) may be any acyclic and cyclic, branched or unbranched, saturated or unsaturated, aliphatic or aromatic hydrocarbon compounds having at least one or more than one thiol group. For example and with preference, thioalcohols used may be aminoalkanethiols, for example aminoethanethiol, aminopropanethiol, aminobutanethiol or aminopentanethiol, or alkylaminoalkanethiols, for example propylaminopropanethiol, propylaminobutanethiol or propylaminoethanethiol, or dialkyl-aminoalkanethiols, for example dimethylaminoethanethiol, or mercaptoalkylamides, for example N-(2-mercapto-ethyl)propionamide, or aminoalkanephosphonates, N-alkyl-N-(mercaptoalkyl)mercapto-alkylanilines, for example N-(2-mercaptoethyl)-4-(2-mercaptoethyl)aniline, N-(2-mercaptoethyl-N-methyl-4-(2-mercaptoethyl)-aniline, N-ethyl-N-(2-mercaptoethyl)-4-(2-mercaptoethyl)aniline, N-(2-mercaptopropyl)-4-(2-mercapto-ethyl)ahiline, N-(2-mercaptopropyl)-N-methyl-4-(2-mercaptoethyl)aniline, N-ethyl-N-(2-mercaptopropyl)-4-(2-mercaptoethyl)aniline, N-(2-mercaptoethyl)-4-(2-mercaptopropyl)-aniline, N-(2-mercaptoothyl)-N-methyl-4-(2-mercaptopropyl)aniline, N-ethyl-N-(2-mercaptoethyl)-4-(2-mercaptopropyl)aniline, N-(2-mercaptopropyl)-4-(2-mercaptopropyl)-aniline, N-(2-mercaptopropyl)-N-methyl-4-(2-mercaptopropyl)aniline, N-ethyl-N-(2-mercaptopropyl)-4-(2-mercaptopropyl)aniline, or mercaptoalkylphenylpyridines, for example 2-(4-mercaptomethylphenyl)pyridine, 3-(4-mercaptomethylphenyl)pyridine, 2-(3-mercapto-methylphenyl)pyridine, 3-(3-mercaptomethylphenyl)pyridine, 4-(3-mercaptomethylphenyl)pyridine, 2-(2-mercaptomethy/phenyl)pyridine, 3-(2-mercaptomethylphenyl)pyridine, 4-(2-mercaptomethylphenyl)pyridine, 2-(4-(2-mercapto-ethyl)phenyl)pyridine, 3-(4-(2-mercaptoethyl)phenyl)pyridine, 4-(4-(2-merceptoethyl)-phenyl)pyridine, 2-(3-(2-mercaptoethyl)phenyl)pyridine, 3-(3-(2-mercapto-5-ethyl)phenyl)-pyridine, 4-(3-(2-mercaptoethyl)phenyl)pyridine, 2-(2-(2-mercaptoethyl)phenyl)pyridine, 3-(2-(2-mercaptoethyl)phenyl)pyridine, 4-(2-(2-mercaptoethyl)phenyl)pyridine, or pyridinealkenethiols, for example 4-pyridinemethanethiol, 3-pyridinemethanethiol, 2-(4-pyridyl)ethanethiol, 2-(2-pyridyl)ethanethiol, 2-(3-pyridyl)ethanethiol, 3-(4-pyridyl)propanethiol, 3-(3-pyridyl)propanethiol, 3-(4-pyridyl)propanethiol, 4-(4-pyridyl)butanethiol, 4-(3-pyridyl)butanethiol, 4-(2-pyridyl)butanethiol, or mercaptoalkylbenzylamines, imidazole alkyl thiols, phthalimidine alkyl thiol, for example s-acetyl-n-(2′-mercaptoethyl)phthalimidine, or aminothiophenols or any desired mixture of these compounds.

Thioesters used may include, for example and with preference, pyridine alkyl thioesters, for example 2-(2′-thioacetateethyl)pyridine, 4-(2′-thioacetateethyl)pyridine, or imidazole alkyl thioesters, for example 2-mercaptoethylbenzimidazole, or phthalimidine alkyl thioesters, for example N,S-diacetyl-2-mercaptoethylbenzimidazole, or mixtures of these compounds.

Thioethers used may include, for example and with preference, pyridine alkyl sulphides, for example 2-(2′-tert-butylthioethyl)pyridine, 4-(2′-tert-butylthioethyl)pyridine, imidazoalkyl sulphide, polysulphur thioalkyl compounds, for example 2-(6′-tert-butylthiohexylthio)-pyridine, 2-(4′-tert-butylthiobutylthio)pyridine, 2-(5′-tert-butylthlopentyithio)pyridine, 2-(3′-tert-butylthiopropylthio)pyridine, 4-(3′-tert-butylthiopropylthio)pyridine, polysulphur thiopyridine, for example 2-(3′-tart-butylthlopropylthioethyl)pyridine, 4-(6′-tert-butylthiohexylthioethyl)pyridine, 4-(4′-tert-5-butylthiobutylthioethyl)pyridine, 4-(5′-tert-butylthiopentylthioethyppyridine, 4-(3′-tert-butylthlopropylthioethyl)pyridine, polysulphur thiobenzothiazole, polysulphur thioimidazole, or thiazolidine or derivatives thereof, for example 3-methylthiazolidine, 2-methyl-2-ethylthiazolidine, 2-methyl-2-dodecyl-thiazolidine, 2-methyl-2-carbethoxymethylthiazolidine, 2,2,4,5-tetramethylthiazolidine, 2,2,3-trimethylthiazolidine, 2,2-dimethyl-3-octylthiazolidine, 2-methyl-2-ethyl-3-aminoethylthiazolidine, 2-cyclohexylthiazolidine, 2,2′-dimethylthiazolidine and any desired mixtures of these compounds.

The sulphur compounds used in step c) may more preferably include aminoalkyl thiols, such as especially aminoethanethiol, aminopropanethiol, aminobutanethiol or aminopentanethiol, or thiazolidine or derivatives thereof, such as especially 3-methylthiazolidine, 2-methyl-2-ethylthiazolidine, 2-methyl-2-dodecylthiazolidine, 2-methyl-2-carbethoxymethylthiazolidine, 2,2,4,5-tetramethylthiazolidine, 2,2,3-trimethylthiazolidine, 2,2-dimethyl-3-octylthiazolidine, 2-methyl-2-ethyl-3-aminoethylthiazolidine, 2-cyclohexylthiazolidine or 2,2⁴-dimethylthiazolidine, or pyridinealkanethiols such as especially 4-pyridinemethanethiol, 3-pyridinemethanethiol, 2-(4-pyridylethanethiol, 2-(2-pyridyl)ethanethiol, 2-(3-pyridyl)-ethanethiol, 3-(4-pyridyl)propanethiol, 3-(3-pyridyl)propanethiol, 3-(4-pyridyl)propanethiol, 4-(4-pyridyl)butanethiol, 4-(3-pyridyl)butanethiol or 4-(2-pyridyl)butanethiol or mixtures of these compounds.

The sulphur compounds used in step c) may most preferably include dimethylthiazolidines, such as especially 2,2′-dimethylthiazolidine, aminoethanethiol and 4-pyridineethanethiol or isomers thereof or mixtures of these compounds.

The sulphur compounds used in step c) can likewise be used in their salt form, i.e., for example, as acid-base adducts in the presence of hydrochloric acid or sulphuric acid or other inorganic or organic acids.

The total amount of strongly acidic groups present in the sulphonated crosslinked bead polymer from step b) may preferably be loaded only partly with the sulphur compounds. Based on the total amount of acidic groups in the bulk of sulphonated crosslinked bead polymer from step b) in mol equated to 100%, between 5 and 45 mol %, preferably between 15 and 30 mol %, of sulphur compounds may be used,

Step c) can be conducted either in a column method or in a batchwise method. In the batchwise method, which may be employed preferentially, the loading may be effected in water or in organic media or in mixtures thereof. Step c) of the process according to the invention may be conducted in such a way, for example, that the sulphonated crosslinked bead polymers from step b) may first be initially charged in water or other organic media or else coming directly from step b) , without further addition of liquids, and then the mixture may be inertized. For example, the sulphonated crosslinked bead polymers from step b) may be inertized by addition of nitrogen or other inert gases, for example argon. For example, the sulphur compound may then be added in step c), for example by metered addition, while stirring, However, it may likewise be possible to add the total amount of the sulphur compound in step c) all at once to the sulphonated crosslinked bead polymer from step b). Preference may be given to metered addition. Thereafter, the mixture can be stirred, for example, for between 30 min and 10 hours. Preferably, the mixture may be stirred for between 2 h and 6 h. For example, the reaction mixture can then be worked up in step c) by adding inertized water. For example, it may be additionally possible to add further inert gas to this mixture. Preferably, the mixture may be inertized by adding nitrogen, but it may also be possible to use other inert gases. Preferably, the mixture may be inertized with the inert gas in step c) for between 1 min and 10 min. The mixture can, however, likewise be inertized with the inert gas for a shorter or longer period,

Preferably, step c) may be conducted in such a way that the sulphonated crosslinked bead polymers may first be initially charged and then inertized by addition of an inert gas. Thereafter, preferably, the sulphur compound may be added by metered addition while stirring. Thereafter, the mixture may be stirred further, preferably for a period of 2 h to 6 h. Then inertized water may preferably be added, Thereafter, further inert gas, preferably nitrogen, may preferably be used for inertization of the mixture in step c).

Step c) may preferably be conducted at temperatures between 5° C. and 80° C., even further preferably at temperatures between 10 and 30° C.

The catalyst prepared in the process according to the invention may preferably be stored under inert gas.

Since the catalyst prepared by the process according to the invention releases a particularly small amount of TOC to an aqueous medium within 20 h, the catalyst having a TOC (total organic carbon) release amount of less than or equal to 3 ppm, preferably between 1 ppm and 3 ppm, may likewise be encompassed by the invention. An aqueous medium in the context of the invention may preferably be demineralized water. The TOC content may be determined in accordance with the invention as follows:

The catalyst may be washed four times with water and, directly after the treatment, introduced into a heatable glass filter column. The temperature of the filter column may be set to 70° C. By means of a peristaltic pump, boiled demineralized water may then be pumped through the ion exchanger at a rate of 0.2 BV/h within a period of 20 h.

The eluate may be captured and collected in portions in glass bottles. In the fourth eluate bed volume captured, the TOC content may be analysed.

The mean bead diameter of the catalysts prepared in accordance with the invention may be between 30 μm and 2000 μm, preferably between 500 and 1000 μm, more preferably between 500 and 800 μm. The catalysts prepared in accordance with the invention can be prepared in heterodisperse or monodisperse form. Preference may be given to preparing monodisperse catalysts. The catalysts prepared in accordance with the invention have gel-like properties and may therefore also be referred to as catalyst gels.

In the present application, “monodisperse” refers to those substances in which at least 90% by volume or by mass of the particles have a diameter within the interval of ±10% of the most common diameter.

For example, in the case of a substance having the most common diameter of 0,5 mm, at least 90% by volume or by mass may be within a size interval between 0.45 mm and 0.55 mm; in the case of a substance having the most common diameter of 0.7 mm, at least 90% by volume or by mass may be within a size interval between 0.77 mm and 0.63 mm.

The catalysts can be used in condensation, addition and esterification reactions, for example, such as those described in DE 10027908 A1, the content of which with regard to these reactions may be encompassed by the present patent application,

Preferably, the catalyst gels may be used in condensation reactions for synthesis of bisphenols proceeding from phenols, o-, m- or p-cresols or alpha- and beta-naphthols and ketones, for example and with preference acetone, acetophenone, butanone, hexafluoroacetone or cyclohexanone, more preferably for synthesis of bisphenol A (2,2-bis(4-hydroxyphenyl)propane (BPA)) from phenol and acetone. The invention therefore likewise encompasses the use of the catalysts prepared in accordance with the invention for preparation of bisphenols from phenols and ketones, preferably for preparation of bisphenol A from phenol and acetone.

By means of the process according to the invention, it may be possible for the first time to prepare bisphenol catalysts, especially bisphenol A catalysts, without using environmentally harmful swelling agents. In addition, it has been found that these catalysts have a particularly high total capacity. Moreover, the process according to the invention enables the preparation of catalysts which have reduced TOC release and may therefore also be preferred from an ecotoxicological point of view for this reason.

### EXAMPLES

### Test Methods

### Determination of the Amount of Acid Released Into the Aqueous Eluate by the Catalyst

A glass column having a base frit may be charged with 50 ml of catalyst together with demineralized water. The water may be released down to the resin bed level. Then a further 10 ml of water are metered in, The resin may be left to stand for 24 hours. Thereafter, the resin may be eluted with demineralized water—flow rate 80 ml per hour. The eluate may be collected in 20 ml portions and titrated with 0.01 molar sodium hydroxide solution.

### Determination of the TOC Content

### Pretreatment

100 ml of resin are shaken in in the H⁺ form under demineralized water. Then the resin may be transferred into a 600 ml beaker and the water may be filtered off with suction. 400 ml of demineralized water are added to the beaker and filtered off with suction again. This operation may be repeated a total of 4 times.

### Testing

Directly after the pretreatment, the pretreated ion exchanger may be introduced into the heatable glass filter column, The temperature of the filter column may be set to 70°C. By means of a peristaltic pump, boiled demineralized water may then be pumped through the ion exchanger at a rate of 0.2 BV/h within a period of 20 h.

The eluate may be captured and collected in portions in glass bottles. In the fourth eluate bed volume captured, the TOG content may be analysed.

The figure is reported in mg TOG per litre of liquid.

### Determination of the Level of the Total Capacity

100 ml of demineralized water are metered into a 200 ml beaker at 25° C.

Into this are metered 20 ml of resin in the hydrogen form. Subsequently, 5 grams of NaCl p.a. are metered in.

The suspension is stirred for 5 minutes. This is followed by titration with 1 n sodium hydroxide solution in a titrator.

The laboratory machine calculates the level of the total capacity in mol of strongly acidic groups per litre of resin via the consumption of sodium hydroxide solution.

### Example 1

### Preparation of a Monodisperse Crosslinked Bead Polymer Gel

A 4 l glass reactor equipped with stirrer, condenser, thermocouple and nitrogen gas feed is initially charged with 1160 ml of deionized water. Into this are metered 3.59 g of boric acid and 0.99 g of sodium hydroxide, which are dissolved.

Dispersed into this solution are 300 grams of a microencapsulated styrene polymer in bead form having a copolymerized divinylbenzene content of 1.0% by weight as seed. The microcapsule wall consists of a formaldehyde-hardened complex coacervate composed of gelatin and an acrylamide/acrylic acid copolymer.

Then, within 30 minutes at room temperature, a mixture of 847 grams of styrene, 48.75 grams of 80% by weight divinylbenzene commercial mixture of divinylbenzene, ethylstyrene and ethylbenzene—and 4.5 grams of Trigonox 21 S is metered in. The suspension is stirred at room temperature for a further 2 hours. Thereafter, within 30 minutes, 100 grams of a 2% by weight aqueous solution of Walocel MT 400 are metered in. The suspension is heated to 63° C. within 90 minutes and stirred at 63° C. for a further 10 hours.

Subsequently, within 60 minutes, the mixture is heated to 95° C. and stirred at 95° C. for a further 2 hours.

After cooling, the suspension is metered into a 10 litre reactor which has been initially charged with 4 litres of demineralized water. The mixture is stirred for 5 minutes. The suspension is poured onto a suction filter. The resultant bead polymer is dried at 70° C. for 4 hours.

Yield of bead polymer after drying: 1193 grams

### Example 2

### Preparation of a Strongly Acidic Cation Exchanger Without Use of the 1,2-Dichloroethane Swelling Agent During the Sulphonation

Apparatus:

3 litre jacketed flange reactor; HP 4 thermostat; precision glass gate stirrer; graduated dropping funnel; solids funnel; measurement data recorder

At room temperature, 845 grams of 98% by weight sulphuric acid are initially charged. The acid is heated to 100° C. Within 15 minutes, 100 grams of monodisperse bead polymer prepared as in example 1 are metered in. The mixture is stirred at a stirrer speed of 150 rpm. Then it is heated to 135° C. within one hour and stirred at this temperature for a further 5 hours.

After cooling to room temperature, the reaction mixture is rinsed out of the reactor into a column with 78% by weight sulphuric acid.

Beginning with 78% by weight sulphuric acid, sulphuric acid of decreasing concentration is filtered through the reaction mixture present in the column. Finally, water is used for filtration.

If the cation exchanger is in water-moist form, one bed volume of demineralized water is filtered at 70° C. within one hour, Thereafter, the cation exchanger is left to stand at 70° C. for 1 hour. Thereafter, within 2 hours, 2 bed volumes of demineralized water are filtered through the cation exchanger at 70° C.

Then the cation exchanger is cooled to room temperature.

Volume yield: 675 ml

Dry weight: 0.2646 grams per mi of cation exchanger

Total capacity of hydrogen form: 1.35 mol/l

Total capacity of sodium form: 1.46 mol/l

A total of 0.4 mmol of acid is eluted per litre of resin.

### Example 3

### Preparation of a Strongly Acidic Cation Exchanger With Use of the 1,2-Dichloroethane Swelling Agent During the Sulphonation

Apparatus:

3 litre jacketed flange reactor; HP 4 thermostat; precision glass gate stirrer; graduated dropping funnel; solids funnel; measurement data recorder

At room temperature, 623 grams of 85% by weight sulphuric acid are initially charged. Within 15 minutes, 100 grams of monodisperse bead polymer prepared as in example 1 are metered in. The mixture is stirred at a stirrer speed of 150 rpm. Within 5 minutes, 79 ml of 1,2-dichloroethane are metered in. It is metered in at 25° C. within 30 minutes. Then 230 grams of 65% by weight oleum are metered in at room temperature within 30 minutes. In the course of this, the temperature rises to 55° C. Then the mixture is heated to 115° C. within one hour and stirred at 115° C. for a further 3 hours. In the course of this, 1,2-dichloroethane is distilled off. Compressed air is passed through, and this drives out remaining 1,2-dichloroethane. Then the mixture is heated to 135° C. within 30 minutes and stirred at this temperature for a further 5 hours.

After cooling to room temperature, the reaction mixture is rinsed out of the reactor into a column with 78% by weight sulphuric acid.

Beginning with 78% by weight sulphuric acid, sulphuric acid of decreasing concentration is filtered through the reaction mixture present in the column. Finally, water is used for filtration.

If the cation exchanger is in water-moist form, one bed volume of demineralized water is filtered at 70° C. within one hour. Thereafter, the cation exchanger is left to stand at 70° C. for 1 hour. Thereafter, within 2 hours, 2 bed volumes of demineralized water are filtered through the cation exchanger at 70° C.

Then the cation exchanger is cooled to room temperature,

Volume yield: 710 ml

Dry weight: 0.2511 grams per ml of cation exchanger

Total capacity of hydrogen form: 1.27 mol/l

Total capacity of sodium form: 1.37 mol/l

### Example 4

### Preparation of a Monodisperse Catalyst by Loading a Strongly Acidic Cation Exchanger With 2,2′-Dimethylthiazoildine

Based on the total amount of add in mol present in the amount of resin used, 20 mol % of 2,2′-dimethylthiazolidine is used.

Apparatus:

3 litre jacketed flange reactor; HP 4 thermostat; precision glass gate stirrer; graduated dropping funnel; solids funnel; measurement data recorder, gas inlet tube

At room temperature, 600 ml of demineralized water are initially charged.

Into this are metered 1000 ml of cation exchanger prepared as in example 2 while stirring. Thereafter, nitrogen is passed through the reaction mixture for 30 minutes. Then, within 30 minutes at room temperature, 29.5 grams of 2,2′-dimethylthiazolidine are metered in, The mixture is stirred at room temperature for a further 4 hours.

The reaction liquor is drawn off. 600 ml of nitrogen-inertized water are metered in. The mixture is stirred for 5 minutes, in the course of which nitrogen is passed through the reaction mixture.

The catalyst is discharged into a nitrogen-flooded glass bottle and sucked dry. For 10 minutes, nitrogen is passed through the reaction mixture.

Dry weight: 26.82 grams per 100 ml of moist catalyst

Total capacity of original form: 1.01 mol/l

Total capacity of sodium form: 1.07 mol/l

### Result

TABLE 1

| Eluate number | Amount of acid in mmol per litre of resin |
|---|---|
| First eluate | 0.08 |
| Second eluate | 0 |

### Example 5

### Preparation of a Monodisperse Catalyst by Loading a Strongly Acidic Cation Exchanger With 2,2′-Dimethylthiazoildine

Based on the total amount of acid in mol present in the amount of resin used, 20 mol % of 2,2′-dimethylthiazolidine is used.

Apparatus:

3 litre jacketed flange reactor; HP 4 thermostat; precision glass gate stirrer; graduated dropping funnel; solids funnel; measurement data recorder, gas inlet tube

At room temperature, 600 ml of demineralized water are initially charged.

Into this are metered 1000 ml of cation exchanger prepared as in example 3 while stirring, Thereafter, nitrogen is passed through the reaction mixture for 30 minutes. Then, within 30 minutes at room temperature, 27.5 grams of 2,2′-dimethylthiazolidine are metered in. The mixture is stirred at room temperature for a further 4 hours.

The reaction liquor is drawn off. 600 ml of nitrogen-inertized water are metered in. The mixture is stirred for 5 minutes, in the course of which nitrogen is passed through the reaction mixture.

The catalyst is discharged into a nitrogen-flooded glass bottle and sucked dry. For 10 minutes, nitrogen is passed through the reaction mixture.

Dry weight: 23.02 grams per 100 ml of moist catalyst

Total capacity of original form: 0.96 mol/l

Total capacity of sodium form: 1.04 mol/l

### Result

TABLE 2

| Eluate number | Amount of acid in mmol per litre of resin |
|---|---|
| First eluate | 0.08 |
| Second eluate | 0.02 |

A total of 0.1 mmol of acid is eluted per litre of resin.

### Monodisperse Cation Exchangers Not Loaded With 2,2′-Dimethylthiszolidine

TABLE 3

| | | Total capacity | Wagner test | | |
|---|---|---|---|---|---|
| Example | Sulphonation | mol/l | Conductivity 4BV | Conductivity 2BV | TOC (elution 4BV) |
| 2 | without 1,2-dichloroethane | 1.35 | 18.74 μS/cm | 26.03 μS/cm | 3.36 ppm |
| | (DCE) | | | | |
| 3 | with 1,2-dichloroethane | 1.27 | 36.94 μS/cm | 70.35 μS/cm | 7.95 ppm |
| | (DCE) | | | | |

### Monodisperse Cation Exchangers Loaded with 2,2′-Dimethylthiazolidine

TABLE 4

| | | Total capacity | Total capacity | |
|---|---|---|---|---|
| | TOC | partial | partial | Amount of acid eluted |
| Example | (elution 4BV) | H form in mol/l | Na form in mol/l | in mmol per litre of catalyst |
| 4 without DCE | 2.87 ppm | 1.10 mol/l | 1.19 mol/l | 0.08 |
| 5 with DCE | 4.33 ppm | 1.01 mol/l | 1.07 mol/l | 0.1 |

What is claimed is:

1. A process for preparing a catalyst, the process comprising:
a) converting monomer droplets of a mixture comprising at least one monoethyienically unsaturated aromatic compound, at least one multiethylenically unsaturated compound, and at least one initiator to a crosslinked bead polymer;
b) mixing the crosslinked bead polymer with sulphuric acid to form a reaction mixture and sulphonating the crosslinked bead polymer at a temperature of 50° C. to 160° C. to produce sulphonated cross-linked bead polymers, wherein the concentration of the sulphuric acid in the reaction mixture is at least 75% by weight, and the reaction mixture comprises 70% to 95% by weight sulphuric acid and 5% to 30% by weight bead polymer, based on the total amount of sulphuric acid and bead polymer, and a sum total of the percentages by weight of sulphuric acid and bead polymer in the reaction mixture is >96% by weight; and
c) reacting the sulphonated crosslinked bead polymers with at least one sulphur compound selected from thioalcohols, thioethers, thioesters, and mixtures thereof.

2. The process according to claim 1, wherein the monoethylenically unsaturated aromatic compound is selected from styrene, α-methylstyrene, vinyltoluene, ethylstyrene, t-butylstyrene, chlorostyrene, bromostyrene, chloromethylstyrene vinylnaphthalene, and mixtures of thereof.

3. The process according to claim 1, wherein the multiethylenically unsaturated aromatic compound is selected from divinylbenzene, divinyltoluene, trivinylbenzene, octadiene, triallyl cyanurate, and mixtures thereof.

4. The process according to claim 1, wherein the monoethylenically unsaturated aromatic compound is styrene and the multiethylenically unsaturated compound is divinylbenzene.

5. The process according to claim 1. further comprising conducting process step a) in the absence of compounds selected from toluene, ethylbenzene, xylene, cyclohexane, octane, isooctane, decane, dodecane, isododecane, methyl isobutyl ketone, ethyl acetate, butyl acetate, dibutyl phthalate, n-butanol, 4-methyl-2-pentanol, n-octanol, and porogens.

6. The process according to claim 1, wherein the sulphuric acid used for step b has a concentration of 92% to 99%.

7. The process according to claim 6, wherein the reaction mixture comprises 70% to 95% by weight of sulphuric add, and 5% to 30% by weight of the bead polymer, wherein a sum total of the percentages by weight of sulphuric acid and bead polymer based on the amount of the reaction mixture is >96% by weight.

8. The process according to claim 7, wherein a sum total of the percentages by weight of sulphuric acid and bead polymer based on the amount of the reaction mixture is >98% by weight.

9. The process according to claim 1, wherein the temperature in step b) during the sulphonation is 90° C. to 140° C.

10. The process according to claim 1, further comprising conducting the sulphonation in step b) for 3 hours to 12 hours.

11. The process according to claim 1, wherein the sulphur compounds are selected from thiazolidine, derivatives of thiazolidine, aminoalkylthiols, derivatized pyridinethiols and mixtures thereof.

12. The process according to claim 1, wherein the sulphur compounds are selected from 2,2′-dimethylthiazolidine, aminoethanol, 4-pyridinethiol, and mixtures thereof.

13. The process according to claim 1, further comprising conducting the reaction in step c) at a temperature of 10° C. to 30° C.

14. The process according to claim 1, wherein:
the monoethylenically unsaturated aromatic compound is selected from styrene, α-methylstyrene, vinyltoluene, ethylstyrene, t-butylstyrene, chlorostyrene, bromostyrene, chloromethylstyrene vinylnaphthalene, and mixtures of thereof;
the multiethylenically unsaturated aromatic compound is selected from divinylbenzene, divinyltoluene, trivinylbenzene, octadiene, triallyl cyanurate, and mixtures thereof;
the concentration of sulphuric add in the reaction mixture is greater than 60% by weight; and
the sulphur compounds are selected from thiazolidine, derivatives of thiazolidine, aminoalkylthiols, derivatized pyridinethiols and mixtures thereof.

15. The process according to claim 14, wherein:
the sulphuric add used for the reaction mixture has a concentration of 92% to 09% by weight, and the concentration of sulphuric add in the reaction mixture is 89% to 96% by weight;
the sum total of the percentages by weight of sulphuric acid and bead polymer in the reaction mixture is >98% by weight;
the temperature in step b) during the sulphonation is 90° C. to 140° C.; and
the process further comprises;
conducting the sulphonation in step b) for 3 hours to 12 hours; and
conducting the reaction in step c) at a temperature of 10° C. to 30° C.

16. The process according to claim 15. wherein:
the monoetnyienicaliy unsaturated aromatic compound is styrene;
the multiethylenically unsaturated compound is divinylbenzene; and
the sulphur compounds are selected from 2,2′-dimethylthiazolidine, aminoethanol, 4-pyridinethiol, and mixtures thereof.

17. The process according to claim 15, wherein:
the process step a) is conducted in the absence of compounds selected from toluene, ethylbenzene, xylene, cyclohexane, octane, isooctane, decane, dodecane, isododecane, methyl isobutyl ketone, ethyl acetate, butyl acetate, dibutyl phthalate, n-butanol, 4-methyl-2-pentanol, n-octanol, and porogens;
the sulphonation in step b) comprises a 2 stage sulphonation wherein the sulphonation is commenced in a first reaction step at a first temperature of 90° C. to 110° C. for 10 min to 60 min, and continued in a second reaction step at a temperature of 120° C. to 140° C. for 3 hours to 7 hours;
the sulphonation is conducted in the absence of a swelling agent;
step c) is conducted at temperatures of 5° C. to 80° C.; and
step c) comprises:
initially charging the sulphonated crosslinked bead polymers;
inertizing the charged sulphonated crosslinked bead polymers by addition of an inert gas;
adding the sulphur compound by metered addition and with stirring to the inertized sulphonated crosslinked bead polymers;
stirring the mixture for 2 h to 6 h to produce a catalyst product;
adding inertized water to the mixture; and
introducing inert as to the mixture, and storing the catalyst product in inert gas.

18. A catalyst prepared by a process according to claim 1, wherein the catalyst is configured to release an amount of less than or equal to 3 ppm of total organic carbon to an aqueous medium within a 20 hour period.

19. A method for preparing bisphenols, the method comprising reacting at least one phenol with at least one ketone in the presence of the catalyst of claim 18 to produce bisphenols.

20. The method of claim 19, wherein the bisphenol is bisphenol A, and the method comprises reacting phenol and acetone in the presence of the catalyst to produce bisphenol A.
